(19) United States
(12) Patent Application Publication
(10) Pub. No.: US 2012/0172329 A1
(43) Pub. Date: Jul. 5, 2012

(54) PHYTOCHEMICAL COMPOSITIONS INCLUDING XANTHONES FOR ANTI-INFLAMMATORY, ANTI-CYTOKINE STORM, AND OTHER USES

(75) Inventor: Prachya Kongtawelert, Chiang Mai (TH)

(73) Assignee: Thailand Excellence Center for Tissue Engineering, District,, Chiang Mai , (TH)

(21) Appl. No.: 13/496,191

(22) PCT Filed: Sep. 14, 2009
(86) PCT No.: PCT/SG2009/000339

§ 371 Date: Mar. 14, 2012

(51) Int. Cl. A61K 31/352; A61K 31/7008; A61P 31/16; A61P 29/00; A61P 31/12; A61K 31/616; A61K 31/737

(52) U.S. Cl. 514/54; 514/455; 514/161; 514/62

(57) ABSTRACT

A composition (e.g., a phytochemical composition) including a predetermined concentration of xanthones, and in certain embodiments predetermined concentrations of both xanthones and sesamin. The composition facilitates at least one of decreasing pro-inflammatory cytokines, increasing anti-inflammatory cytokines, enhancing a connective tissue anti- degeneration effect, and inhibiting viral neuraminidase (e.g. neuraminidase of an influenza A virus). The composition can further decrease at least one of gene expression and release of pro-inflammatory cytokines. The phytochemical composition can increase at least one of gene expression and release of anti-inflammatory cytokines. The phytochemical composition provides at least one of an anti-inflammatory, an anti-viral (e.g., anti-influenza), and a connective tissue anti-degradation effect in a living organism. A use of xanthones for manufacturing phytochemical compositions that include predetermined concentrations of xanthones is also provided. A use of xanthones in combination with sesamin for manufacturing phytochemical compositions that include predetermined concentrations of both xanthones and sesamin is additionally provided.

### TECHNICAL FIELD

The present disclosure relates generally to phytochemical compositions that include xanthones. More specifically, the present disclosure relates to phytochemical compositions including xanthones that display anti-inflammatory, anti-cytokine storm, and other uses.

### BACKGROUND

A body's immune system and immune cells are essential for fighting pathogens, for example bacteria, viruses, and other foreign matter, that invade or are introduced within the body. Recognition of pathogens within the body typically triggers an immune response. Generally, the immune response involves a production of cytokines. It is generally important that the production of cytokines is well regulated for maintaining homeostatic balance within the body. An imbalance in the production of cytokines, for example an excessive production of cytokines, in the body can cause significant damage to body tissues and organs.

A cytokine storm (which is also known as hypercytokinemia) is a significant immune response to pathogens that invade the body. The precise causation of cytokine storms within the body has not been definitively established. A possible causation of cytokine storms is an encounter, by the immune system, of a new and highly pathogenic pathogen. Cytokine storms are also associated with a number of infectious and non-infectious diseases, including influenza, adult respiratory distress syndrome (ARDS), and systemic inflammatory response syndrome (SIRS). It has been suggested that the influenza A (H1N1) virus triggers cytokine storms within the body.

During a cytokine storm, inflammatory mediators, for example pro-inflammatory cytokines such as Interleukin-1 (IL1), Interleukin-6 (IL6), tumor necrosis factor-alpha (TNF-60 alpha), oxygen free radicals, and coagulation factors are released by the immune cells of the body. Cytokine storms have the potential to cause significant damage to body tissues and organs. For example, occurrence of cytokine storms in the lungs can cause an accumulation of fluids and immune cells, for example macrophages, in the lungs, and eventually block off the body's airways thereby resulting in respiratory distress and even death.

It is generally important to be able to prevent, control, or mitigate the occurrence of the cytokine storm. There are existing or conventional techniques associated with preventing, controlling, or mitigating the occurrence of the cytokine storm (and unwanted inflammation in general). For example, it has been reported that TNF inhibitors may be useful for facilitating the control of cytokine storms and for reducing adverse reactions caused by the occurrence of cytokine storms within the body. In addition, research has suggested that angiotensin converting enzyme (ACE) inhibitors, and angiotensin II receptor blockers (ARBs), may have clinical utility for controlling or down-regulating cytokine storms, and for reducing inflammation, within the body. Corticosteriods and non-steroidal anti-inflammatory drugs (NSAIDS) have also been employed in an attempt to treat patients experiencing cytokine storms. In addition, suggested therapeutic agents for treating influenza-type viral infections include antibodies to the influenza virus neuraminidase.

However, the effectiveness and safety of many conventional techniques associated with the prevention, control, or mitigation of cytokine storms within the body have not been comprehensively or adequately verified. In addition, many conventional anti-influenza treatments (e.g. anti-viral or anti-influenza drugs) have associated undesirable side effects when consumed by a patient. Such undesirable side effects include nausea, vomiting, and toxicity. Furthermore, there is an increasing problem of developed resistance to many conventional anti-viral (e.g. anti-influenza) drugs.

Accordingly, new or enhanced approaches for at least one of preventing, controlling, or mitigating cytokine storms may be useful for improving public health. Furthermore, new compositions or techniques providing anti-influenza or anti-inflammatory properties may also be useful for improving public health.

### SUMMARY

In accordance with a first aspect of the present disclosure, there is disclosed a composition including a concentration of xanthones of substantially between approximately 0.001 ug/ml and approximately 1.0 ug/ml, the composition for at least one of facilitating and effectuating decrease in quantity of a pro-inflammatory cytokine within a living organism when consumed thereby.

In accordance with a second aspect of the present disclosure, there is disclosed a use of xanthones in the manufacture of a composition including xanthones of a concentration of between approximately 0.005 ug/ml and approximately 0.5 ug/ml to therapeutically affect a pro-inflammatory cytokine condition within a living organism when the composition is consumed thereby.

In accordance with a third aspect of the present disclosure, there is disclosed a use of xanthones in combination with sesamin for manufacturing a composition including xanthones at a concentration of between approximately 0.001 ug/ml and approximately 1.0 ug/ml and sesamin at a concentration of at least approximately 0.001 mg/ml to therapeutically affect a pro-inflammatory cytokine condition within a living organism when the composition is consumed thereby.

In accordance with a fourth aspect of the present disclosure, there is disclosed a process for manufacturing a composition that therapeutically affects a pro-inflammatory cytokine condition within a living organism when consumed thereby. The process includes providing the composition with xanthones of a concentration of between approximately 0.005 ug/ml and approximately 0.5 ug/ml.

In accordance with a fifth aspect of the present disclosure, there is disclosed a composition including a predetermined concentration of xanthones to provide a xanthones dosage of at least approximately 5 mg/day when consumed by a living organism, the composition at least one of facilitating and effectuating decrease in quantity of a pro-inflammatory cytokine within the living organism when consumed thereby.

### DETAILED DESCRIPTION

A cytokine storm (also known as hypercytokinemia) is a significant immune response within the body that typically occurs during the course of a number of infectious and non-infectious diseases, for example influenza and acute respiratory distress syndrome. During the cytokine storm, pro-inflammatory cytokines and pro-inflammatory mediators are produced and released by immune cells within the body. Cytokine storms have the potential to cause significant damage to body tissues and organs. The effectiveness and safety of many conventional compositions, methods, and/or processes used for preventing, controlling, down-regulating, and/or terminating cytokine storms within the body have not been comprehensively or adequately verified. Many conventional compositions providing anti-viral (e.g., anti-influenza) or anti-inflammatory effects have associated undesirable side effects when consumed by a patient. Such undesirable side effects include nausea, vomiting, and toxicity. In addition, there is an increasing problem of the development of resistance to many conventional anti-viral (e.g., anti-influenza) drugs. Accordingly, there is a need for new compositions that facilitates or effectuates prevention, control, down-regulation, and/or termination of cytokine storms, including compositions supporting, facilitating, or having anti-viral (e.g., anti-influenza) or anti-inflammatory effects or uses, and which reduce or minimize a likelihood that undesirable side effects or the development of resistance will occur.

Embodiments of the present disclosure are directed to compositions (e.g., phytochemical compositions) that include xanthones, methods and processes for manufacturing said compositions (e.g., phytochemical compositions), and uses of xanthones for the manufacture of said compositions (e.g., phytochemical compositions). In the context of the present disclosure, the term phytochemical shall refer to any compound or chemical that occur naturally in plants (i.e. living organisms belonging to the kingdom Plantae), or to any artificial or synthetic compound, substance, or chemical having an identical or almost identical chemical, physical, and/or structural properties to that which occurs naturally in plants, and the term phytochemical composition shall refer to a composition including at least one phytochemical. Unless defined otherwise, all technical and scientific terms used herein have the same meanings as commonly understood by a person of ordinary skill in the relevant art of the present disclosure.

As mentioned above, embodiments of the present disclosure relate to compositions (e.g., phytochemical compositions) that include predetermined concentrations of xanthones. In some embodiments, the predetermined concentrations of xanthones are provided by extracts from a fruit of a mangostana L. plant (also known as a mangosteen fruit). Extracts from the mangosteen fruit can be obtained using methods known to a person having ordinary skill in the art.

In numerous embodiments, the phytochemical composition further includes a predetermined concentration of sesamin. In several embodiments, the phytochemical composition also includes predetermined concentrations of compositions, substances, or chemicals having anti-viral (e.g., anti-influenza) and/or anti-inflammatory properties.

In most embodiments, the phytochemical composition therapeutically affects a pro-inflammatory cytokine condition, for instance by facilitating or effectuating a decrease or reduction in a quantity of pro-inflammatory cytokines or pro-inflammatory mediators, within biological tissue (e.g., a body of a living organism when consumed thereby). In the context of the present disclosure, the term living organism refers to human beings and animals (i.e., organisms from the kingdom Animalia).

Examples of pro-inflammatory cytokines or pro-inflammatory mediators include interleukin-1alpha (IL-1α) and interleukin-1beta (IL-1β) (hereinafter collectively referred to as interleukin-1 or IL-1), interleukin-6 (IL-6), interluekin-8 (IL-8), interleukin-11 (IL-11), interleukin-12 (IL-12), interleukin-17 (IL-17), interleukin-18 (IL-18), tumor necrosis factor-alpha (TNF-α), interferon-gamma (IFNγ), granulocyte-macrophage colony stimulating factor (GM-CSF), and transforming growth factor-beta (TGF-β). It will be understood by a person having ordinary skill in the art that references to pro-inflammatory cytokines in most embodiments of the present disclosure can refer any one or more of pro-inflammatory cytokines known in the art, and including one or more of the above-listed examples of pro-inflammatory cytokines.

In some embodiments, the decrease in quantity of pro-inflammatory cytokines within the living organism helps to prevent, control, down-regulate, and/or stop the occurrence of a cytokine storm within the living organism. This is to say, in some embodiments, the phytochemical composition provides an anti-cytokine storm effect or function when consumed by the living organism. In several embodiments, the decrease in quantity of pro-inflammatory cytokines within the living organism helps to prevent, control, down-regulate, and/or stop inflammation within the living organism. This is to say, in several embodiments, the phytochemical composition facilitates or provides an anti-inflammatory effect or function when consumed by the living organism. In numerous embodiments, the decrease in quantity of pro-inflammatory cytokines within the living organism contributes to an anti-viral (e.g., anti-influenza) or viral modulatory effect or function (e.g., viral activity inhibitory effect) within the living organism.

In some embodiments, the phytochemical composition therapeutically affects an anti-inflammatory cytokine, anti-inflammatory mediator, or anti-inflammatory factor condition, for instance by facilitating or effectuating increase in a quantity of the anti-inflammatory cytokine, anti-inflammatory mediator, and/or anti-inflammatory factor within the living organism. Examples of anti-inflammatory cytokines, anti-inflammatory mediators, and/or anti-inflammatory factors include interleukin-2 (IL-2), interluekin-4 (IL-4), interleukin-13 (IL-13), and interferon-alpha (IFN-α). A person having ordinary skill in the art will understand that a reference to anti-inflammatory cytokines, anti-inflammatory mediators, and/or anti-inflammatory factors in most embodiments of the present disclosure can relate to any one or more of anti-inflammatory cytokines, anti-inflammatory mediators, and/or anti-inflammatory factors known in the art, which includes the above-listed examples.

In some embodiments, the phytochemical composition facilitates or effectuates inhibition of a viral neuraminidase (e.g., a decrease in an action of viral neuraminidase). In numerous embodiments, the viral neuraminidase is a neuraminidase of a virus contributing to occurrence of cytokine storms within the body of the living organism. In several embodiments, the viral neuraminidase is a neuraminidase of an influenza virus (e.g., influenza virus A, influenza virus B, and influenza virus C). In selected embodiments, the viral neuraminidase is an influenza A (H1N1) neuraminidase virus. In other embodiments, the viral neuraminidase is an influenza A (H2N2), influenza A (H2N1), influenza A (H3N2), influenza A (H5N1), or influenza A (H1N2) neuraminidase virus.

In some embodiments of the present disclosure, the phytochemical composition facilitates or effectuates a decrease in gene expression of one or more pro-inflammatory cytokines within the body. For example, in several embodiments, the phytochemical composition facilitates or effectuates decrease in IL-1 gene expression of an immune cell (i.e. a cell that is involved in immune responses) of the body. Examples of immune cells include lymphocytes, phagocytic cells, fibroblasts, monocytes, neutrophils, and macrophages.

In some embodiments of the present disclosure, the phytochemical composition facilitates or effectuates a decrease in secretion or release of one or more pro-inflammatory cytokines by an immune cell within the body. For example, in several embodiments, the phytochemical composition facilitates or effectuates a decrease in IL-1 release or secretion from the immune cell.

In numerous embodiments, the decrease in gene expression of the pro-inflammatory cytokine (e.g., gene expression of IL-1) results in the decrease of secretion of the pro-inflammatory cytokine (e.g., decrease secretion of IL-1) by the immune cell, and hence a lower quantity of pro-inflammatory cytokine (e.g., lower quantity of IL-1) within the body. The lower quantity of pro-inflammatory cytokines (e.g., IL-1) within the body facilitates or effectuates or provides an anti-inflammatory effect.

In some embodiments of the present disclosure, the phytochemical composition facilitates or effectuates an increase in gene expression of one or more anti-inflammatory cytokine within the body. For example, in several embodiments, the phytochemical composition facilitates or effectuates increased gene expression of IL-2 by the immune cells of the body. In some embodiments of the phytochemical composition facilitates or effectuates an increase in secretion or release of one or more anti-inflammatory cytokine by immune cells within the body. For example, in several embodiments, the phytochemical composition facilitates or effectuates increased secretion of IL-2 by the immune cells of the body.

In numerous embodiments, the increase in the gene expression of one or more anti-inflammatory cytokines (e.g., IL-2) results in the increased secretion of the one or more anti-inflammatory cytokines by the immune cells of the body, and therefore a higher quantity of anti-inflammatory cytokines within the body. The higher quantity of anti-inflammatory cytokines within the body facilitates or effectuates or provides an anti-inflammatory effect.

Several embodiments of the present disclosure are directed to phytochemical compositions that include predetermined concentrations of xanthones for therapeutically affecting a pro-inflammatory cytokine condition, for instance by facilitating or effectuating decrease in quantity of a pro-inflammatory cytokine, therapeutically affecting an anti-inflammatory cytokine condition, for instance by facilitating or effectuating increase in quantity of an anti-inflammatory cytokine, and/or inhibiting a viral neuramidase (e.g., the neuraminidase of the influenza virus) within the living organism. Selected embodiments of the present disclosure are directed to phytochemical compositions that include predetermined concentrations of xanthones in combination with sesamin for facilitating or effectuating any combination of one or more of decrease or reduction in quantity of a pro-inflammatory cytokine, increase in quantity of an anti-inflammatory cytokine, and inhibition of a viral neuramidase (e.g., the neuraminidase of the influenza virus) within the living organism.

Several embodiments of the present disclosure are directed to phytochemical compositions that include predetermined concentrations of xanthones for facilitating or effectuating at least one of prevention, control, down-regulation, or termination of cytokine storms caused or triggered by viral infections (e.g., infections by the influenza viruses, including influenza A (H1N1), influenza A (H2N1), influenza A (H2N2), influenza A (H1N2), influenza A (H2N3), and influenza A (H5N1) viruses. In addition, selected embodiments of the present disclosure relates to phytochemical compositions that include predetermined concentrations of xanthones and sesamin for facilitating or effectuating at least one of prevention, control, down-regulation, or termination of cytokine storms caused or triggered by viral infections (e.g., infection by the influenza viruses, including (H1N1), influenza A (H2N1), influenza A (H2N2), influenza A (H1N2), influenza A (H2N3), and influenza A (H5N1) viruses.

In some embodiments of the present disclosure, particular phytochemical compositions that include predetermined concentrations of xanthones, or predetermined concentrations of xanthones and sesamin, facilitate or effectuate an anti-inflammatory, anti-degeneration, or ECM preservation effect upon connective tissue. In the context of the present disclosure, the term “connective tissue” encompasses connective tissue proper (e.g., dense or fibrous connective tissue), as well as specialized connective tissue such as cartilage or bone. Additionally, in several embodiments of the present disclosure, combinations of particular phytochemical compositions with anti-inflammatory substances or compositions provide an enhanced or synergistic anti-inflammatory, anti-degeneration, or ECM preservation effect upon connective tissue as compared to the anti-inflammatory substance or composition alone.

In many embodiments of the present disclosure, the concentration of xanthones within the phytochemical composition can be varied as required. In many embodiments, the concentration of xanthones within the phytochemical composition is selected or determined for providing the living organism with a xanthones dosage of at least approximately 5 milligrams (mg) per day when consumed by the living organism. In some embodiments, the concentration of xanthones within the phytochemical composition is selected or determined for providing the living organism with a xanthones dosage of at least approximately 10 mg per day when consumed by the living organism. In several embodiments, the concentration of xanthones within the phytochemical composition is selected or determined for providing the living organism with a xanthones dosage of at least approximately 20 mg per day when consumed by the living organism.

In most embodiments of the present disclosure, the predetermined concentration of xanthones of the phytochemical composition can be selected and varied as required. In many embodiments, the predetermined concentration of xanthones within the phytochemical composition is substantially between approximately 0.0005 ug/ml and approximately 1.5 ug/ml. In some embodiments, the predetermined concentration of xanthones within the phytochemical composition is substantially between approximately 0.001 ug/ml and approximately 1.0 ug/ml.

As described above, phytochemical compositions provided by some embodiments of the present disclosure includes predetermined concentrations of both xanthones and sesamin. In several embodiments, the concentration of sesamin can be varied as required. In numerous embodiments, the concentration of sesamin of the phytochemical composition is selected or determined for providing the living organism with a sesamin dosage of at least approximately 5 mg per day when consumed by the living organism. In various embodiments, the concentration of sesamin of the phytochemical composition is selected for providing the living organism with a sesamin dosage of at least approximately 10 mg per day when consumed by the living organism. In certain embodiments, the concentration of sesamin of the phytochemical composition is selected or determined for providing the living organism with a sesamin dosage of at least approximately 20 mg per day when consumed by the living organism.

In some embodiments of the present disclosure, the phytochemical composition includes a concentration of sesamin of substantially between approximately 0.01 mM and approximately 2.0 mM. In some embodiments, the phytochemical composition has a concentration of sesamin of substantially between approximately 0.025 mM and approximately 1.0 mM. In selected embodiments, the phytochemical composition has a concentration of sesamin of substantially between approximately 0.10 mM and approximately 0.50 mM.

In many embodiments of the present disclosure, the phytochemical composition is or forms a portion of a food product, a beverage product, or a drug or pharmaceutical product. In other embodiments of the present disclosure, the phytochemical composition is a supplement, additive, or ingredient for one of the food product, the beverage product, and the drug, pharmaceutical, or other consumable (e.g., ingestible) product. It will be understood by a person having ordinary skill in the art that the phytochemical composition can be in various forms or formulations, for example a powder, a paste, or an emulsion.

When particular phytochemical compositions of the present disclosure form portions of beverage products, the xanthones concentrations and any associated sesamin concentrations of such phytochemical compositions can be varied as required to correspondingly vary the xanthones concentrations and sesamin concentrations of the beverage products. For example, in some embodiments, each of the xanthones and sesamin concentration in the beverage product is at least approximately 1.0 mg/liter of the beverage product. In other embodiments, each of the xanthones and sesamin concentration in the beverage product is at least approximately 5.0 mg/liter or approximately 10 mg/liter of the beverage product.

When particular phytochemical compositions of the present disclosure form portions of food or other consumable products, the xanthones concentrations and any associated sesamin concentrations of such phytochemical compositions can be varied as required to vary the xanthones concentrations and sesamin concentrations of the food or other consumable product. In some embodiments, each of the xanthones and sesamin concentration in the food or other consumable product is at least approximately 1.0 mg/kilogram of the food or other consumable product. In other embodiments, each of the xanthones and sesamin concentration in the food or other consumable product is at least approximately 5.0 mg/kilogram or approximately 10 mg/kilogram of the food or other consumable product.

In some embodiments of the present disclosure, particular phytochemical compositions as disclosed above can be combined, mixed, synthesized, or manufactured with other drugs, pharmaceutical compositions, phytochemical compositions, and/or nutraceutical compositions. In several embodiments, the phytochemical compositions as disclosed above can be combined, mixed, synthesized, or manufactured with anti-viral and/or anti-inflammatory drugs, pharmaceutical compositions, phytochemical compositions, and/or nutraceutical compositions. In selected embodiments, the phytochemical compositions as disclosed above can be combined, mixed, synthesized, or manufactured with an anti-inflammatory drug, for example, ibuprofen, diclofenac, aspirin, or naproxen. Additionally or alternatively, a phytochemical composition according to the present disclosure can include or be combined, mixed, synthesized, or manufactured with another substance or composition associated with anti-inflammatory activity; or a substance associated with connective tissue (e.g., connective tissue proper, cartilage, and/or bone) maintenance, support, or regeneration, such as one or more of a glucosamine compound (e.g., glucosamine sulfate or glucosamine hydrochloride), a chondroitin compound (e.g., chondroitin sulfate), methylsulfonylmethane (MSM), or an omega-3 fatty acid. In other embodiments, the phytochemical compositions as disclosed above can be combined, mixed, synthesized, or manufactured with an anti-viral drug, for example oseltamivir (Tamiflu) and zanamivir (Relenza). Additionally or alternatively, a phytochemical composition in accordance with the present disclosure can be combined, mixed, synthesized, or manufactured with a cholesterol lowering drug such as a statin.

In some embodiments, the combination or mix of a particular phytochemical composition provided by the present disclosure with a particular drug, pharmaceutical composition, phytochemical composition, and/or nutraceutical composition increases or enhances the effectiveness, action, and/or safety of the particular drug, pharmaceutical composition, phytochemical composition, and/or nutraceutical composition, for example, by way of a synergistic effect. In several embodiments, the combination of a phytochemical composition provided by the present disclosure with a particular anti-viral (e.g., anti-influenza) and/or anti-inflammatory drugs, pharmaceutical compositions, phytochemical compositions, and/or nutraceutical compositions increases or enhances the anti-viral (e.g., anti-influenza) and/or anti-inflammatory effects or actions of the said anti-viral (e.g., anti-influenza) and/or anti-inflammatory drugs, pharmaceutical compositions, phytochemical compositions, and/or nutraceutical compositions, respectively.

Uses of xanthones for the manufacture of certain phytochemical compositions disclosed above are also provided by embodiments of the present disclosure. Many embodiments of the present disclosure provide uses of xanthones for the manufacture of phytochemical compositions that include predetermined concentrations of xanthones for facilitating or effectuating at least one of decreasing a quantity of pro-inflammatory cytokines, increasing a quantity of anti-inflammatory cytokines, and inhibiting viral neuraminidase (e.g., influenza A viral neuraminidase) within the living organism. In addition, uses of xanthones in combination with sesamin for the manufacture of certain phytochemical compositions disclosed above are also provided by embodiments of the present disclosure. Some embodiments of the present disclosure provides uses of xanthones in combination with sesamin for the manufacture of phytochemical compositions that include predetermined concentrations of both xanthones and sesamin for facilitating or effectuating at least one of decreasing a quantity of pro-inflammatory cytokines, increasing a quantity of anti-inflammatory cytokines, and inhibiting viral neuraminidase (e.g., influenza A viral neuraminidase) within the living organism.

Several embodiments of the present disclosure provide uses of xanthones for the manufacture of phytochemical compositions that include predetermined concentrations of xanthones for decreasing at least one of pro-inflammatory cytokine gene expression and pro-inflammatory cytokine release by the immune cell. Numerous embodiments of the present disclosure provide uses of xanthones for the manufacture of phytochemical compositions that include predetermined concentrations of xanthones for increasing at least one of anti-inflammatory cytokine gene expression and anti-cytokine release by the immune cell. In addition, various embodiments of the present disclosure provide uses of xanthones in combination with sesamin for the manufacture of phytochemical compositions that include predetermined concentrations of both xanthones and sesamin for decreasing at least one of pro-inflammatory cytokine gene expression and pro-inflammatory cytokine release by the immune cell. Selected embodiments of the present disclosure provide uses of xanthones in combination with sesamin for the manufacture of phytochemical compositions that include predetermined concentrations of both xanthones and sesamin for increasing at least one of anti-inflammatory cytokine gene expression and anti-cytokine release by the immune cell.

Processes and methods for manufacturing particular phytochemical compositions as disclosed above are also provided by embodiments of the present disclosure. In multiple embodiments, the process for manufacturing the phytochemical composition includes providing a predetermined concentration of xanthones such that the phytochemical composition is able to provide the living organism with a xanthone dosage of at least approximately 5 mg per day. In some embodiments, the process for manufacturing the phytochemical composition includes providing a predetermined concentration of xanthones such that the phytochemical composition is able to provide the living organism with a xanthones dosage of at least approximately 10 mg per day. In several embodiments, the process for manufacturing the phytochemical composition includes providing a predetermined concentration of xanthones such that the phytochemical composition is able to provide the living organism with a xanthones dosage of at least approximately 20 mg per day.

In numerous embodiments, the process for manufacturing the phytochemical composition further includes providing a predetermined concentration of sesamin such that the phytochemical composition is able to further provide the living organism with a sesamin dosage of at least approximately 5 mg per day. In selected embodiments, the process for manufacturing the phytochemical composition further includes providing a predetermined concentration of sesamin such that the phytochemical composition is able to further provide the living organism with a sesamin dosage of at least approximately 10 mg per day. In specific embodiments, the process for manufacturing the phytochemical composition further includes providing a predetermined concentration of sesamin such that the phytochemical composition is able to further provide the living organism with a sesamin dosage of at least approximately 20 mg per day.

In several embodiments, the process for manufacturing the phytochemical composition further includes combining, mixing, synthesizing or manufacturing the phytochemical composition with existing drugs, pharmaceutical compositions, phytochemical compositions, and/or nutraceutical compositions. In several embodiments, the process for manufacturing the phytochemical composition according to the present disclosure can include combining, mixing, synthesizing or manufacturing the phytochemical composition with another substance or composition associated with anti-inflammatory activity (e.g., ibuprofen, diclofenac, aspirin, or naproxen); or a substance associated with connective tissue, cartilage, or bone maintenance, support, or regeneration, such as one or more of a glucosamine compound (e.g., glucosamine sulfate or glucosamine hydrochloride), a chondroitin compound (e.g., chondroitin sulfate), methylsulfonylmethane (MSM), or an omega-3 fatty acid. In numerous embodiments, the process for manufacturing the phytochemical composition according to the present disclosure can include combining, mixing, synthesizing or manufacturing the phytochemical composition with an anti-viral drug (e.g., oseltamivir (Tamiflu) and zanamivir (Relenza)), and/or a cholesterol lowering drug such as a statin.

For increased clarity and understanding of the present disclosure, representative examples of phytochemical compositions, and effects or properties of such phytochemical compositions, are included in the following description. In addition, for the convenience of the reader, general discussions of xanthones, sesamin, pro-inflammatory cytokines, anti-inflammatory cytokines, viral neuraminidase, and influenza A (H1N1) are also provided in the following disclosure prior to the description of representative examples. It will be understood by a person of ordinary skill in the art that the details presented in the specific examples of the present disclosure are for clarity of illustration and increased understanding, and do not limit the scope of the present disclosure.

### General Discussion of Xanthones

Xanthones are of a class of phytochemicals or active compounds present in extracts from a fruit of Garcinia mangostana L. (also known as a mangosteen fruit). Garcinia mangostana L. is from a family of plants known as Guttiferae. Garcinia mangostana L. has been cultivated for a considerable time in several tropical areas of the world.

Extracts from the mangosteen plant can be obtained using a variety of devices, methods, processes, and techniques known to a person skilled in the relevant art. A variety of solvents, for example water, alcohol, or other edible solvents with sufficient polarity can be used for extracting the extracts of the mangosteen fruit. In one process of extracting mangosteen juice, the mangosteen fruit is first either hand picked or picked by mechanical equipment. The mangosteen fruit is then thoroughly cleaned and allowed to ripen before any processing occurs. When ready for processing, the mangosteen fruit is first inspected for spoilage or for an excessively green color and hardness. A manual or mechanical separator processes the mangosteen fruit, where the seeds and peel are separated from the juice and pulp. The juice and pulp can be further processed by separating the pulp from the juice through use of filtering equipment. The filtering equipment includes, but is not limited to, a centrifuge decanter, a screen filter, a filter press, reverse osmosis filtration, and any other standard commercial filtration devices known to one of ordinary skill in the art.

A xanthone has a molecular formula C₁₃H₈O₂. The general structure of a xanthone is:

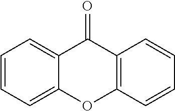

The chemical structure of xanthone forms the central core of xanthones found in natural sources. Xanthones are formed naturally by a condensation reaction of a phenylpropanoid precursor with two malonyl coenzyme A units. Over 200 xanthones have been identified to date. Pharmacological and chemical studies on xanthones suggest that xanthones may possess significant pharmacological properties, for example antidepressant, antitubular, antimicrobial and antiallergenic activities and actions. However, there is still to date a lack of concrete evidence or substantive experimental results for many of the purported health benefits of phytochemicals that are obtained from the mangosteen fruit.

The mangosteen fruit comprises a number of naturally occurring xanthones. For example, the rind of a partially ripen mangosteen fruit yields a polyhydroxy-xanthone derivative, which is termed mangostin, and more specifically beta-mangostin. The rind of fully ripened mangosteen fruit comprises gartanin, 8-disoxygartanin, and normangostin. In addition, an extract from the pericarp of the mangosteen fruit has been reported to have a free radical scavenging effect. Several studies have suggested that a mangostin derivative called gamma-mangostin was found to directly inhibit cyclooxygenase (COX) enzyme activity in mammal glioma cells. The COX enzyme catalyzes the first step in the creation of prostaglandins from a common fatty acid. More specifically, the COX enzyme adds two oxygen molecules to arachidonic acid to begin a set of reactions that ultimately creates a host of free radicals.

For the purposes of the present disclosure, the term “xanthones” used in the present disclosure and in the claims collectively refers to the different types or varieties of xanthones that are present in the extract obtained from the mangosteen fruit.

### General Discussion of Sesamin

Sesamin is a constituent of sesame oil with an epimer or stereoisomer known as episesamin. The chemical structure of sesamin is:

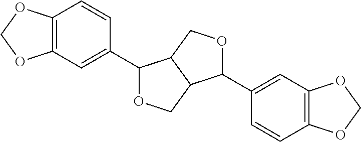

Sesamin is a lignan that is can be extracted from both the bark of Fagara plants and sesame oil. Plant lignans such as sesamin are generally polyphenolic substances that are derived from the phenylalanine amino acid via a dimerization of substituted cinnamic alcohols to a dibenzylbutane skeleton. This dimerization reaction is catalyzed by oxidative enzymes and is typically controlled by dirigent proteins. The term “sesamin” as used in the present disclosure can collectively refer to both sesamin and episesamin.

### General Discussion of Pro-Inflammatory Cytokines

A pro-inflammatory cytokine or a pro-inflammatory mediator is an immuno-regulatory cytokine that favor inflammation. Pro-inflammatory cytokines that are generally responsible for early immune responses include IL-1, IL-6, and TNF-α. IL-1, IL-6, and TNF-α are also considered endogenous pyrogens as they contribute to increasing body temperature. Other examples of pro-inflammatory cytokines or pro-inflammatory mediators include IL-8, IL-11, IL-12, IL-18, GM-CSF, IFN-γ, TGF-β, leukemia inhibitory factors (LIF), oncostatin M (OSM), and a variety of chemokines that attract inflammatory cells.

A pro-inflammatory cytokine generally up-regulates or increases the synthesis of secondary pro-inflammatory mediators and other pro-inflammatory cytokines by immune cells. In addition, pro-inflammatory cytokines can stimulate production of acute phase proteins that mediate inflammation and attract inflammatory cells.

IL-1 is an important pro-inflammatory cytokine. IL-1 is a soluble protein having a mass of approximately 17 kilo-Daltons (kD). IL-1 is produced by a variety of cells, for example macrophages, white blood cells, lymphocytes, monocytes, dendritic cells, and accessory cells that are involved in activation of T-lymphocytes and B-lymphocytes. IL-1 is typically released by such cells during an immune response. IL-1 is generally considered to be a pro-inflammatory cytokine. Pro-inflammatory cytokines generally refer to immuno-regulatory cytokines that favor inflammation.

The original members of the IL-1 superfamily are interleukin-1 alpha (IL-1α), interleukin-1 beta (IL-1β), and interleukin-1 receptor antagonist (IL-IRA). Both IL-1α and IL-1β play important roles in the inflammatory response of the body against pathogens or infection. Both IL-1α and IL-1β recognize a same IL-1 receptor and perform similar biological functions. IL-1α is predominantly a cell-associated molecule whereas IL-1β is generally a secreted molecule. The term “IL-1” used in the present disclosure can be taken to include one or both of IL-1α and IL-1β.

The cytokines or interleukins of the IL-1 superfamily (e.g. IL-1α and IL-1β) are generally produced as precursor peptides, which are then processed to become mature proteins (or mature interleukins). For example, each of IL-1α and IL-1β is produced as a precursor peptide. The precursor peptide of each of IL-1α and IL-1β is then processed by specific enzymes to release a smaller active molecule, which is the mature protein. The three dimensional (3D) structure of mature interleukins of the IL-1 superfamily include twelve to fourteen beta (β) strands or beta sheets (which is a secondary structure of proteins) to form barrel-shaped proteins.

IL-1β (also referred to as IL-1B) is encoded by the IL1B gene. Expression of the IL1B gene produces IL-1β precursor peptide. The IL-1β precursor peptide is first cleaved by an enzyme known as caspase 1 or interleukin-1 beta convertase. The product of this reaction is further cleaved by an enzyme known as cytosolic thiol protease to form mature IL-1β. IL-1α (also referred to as IL-1A) is synthesized primarily as a 31 kDa precursor that lacks a signal peptide. The IL-1α precursor is cleaved by a cysteine protease calpain to form mature IL-1α.

IL-1 is produced during immune responses. A common function of IL-1 (e.g. IL-1α and IL-1β) is an increasing of expression of adhesion factors on endothelial cells to enable transmigration of leukocytes (which are immune cells that fight pathogens) to sites of infection. In addition, IL-1 stimulates the hypothalamus thermoregulatory center to cause an increase in body temperature (i.e. a fever). The increased body temperature helps the body's immune system to fight pathogens or infection within the body. In addition, IL-1β is an important mediator of inflammatory response, and is also involved in a range of cellular activities, for example cell proliferation, cell differentiation, and cell apoptosis. Furthermore, IL-1β has also been found to contribute to the sensation of pain during inflammation.

TNF-α is also an important pro-inflammatory cytokine. TNF-α is involved in systemic inflammation and works in tandem with a variety of other cytokines to stimulate the acute phase immune reaction. TNF-α is capable of inducing apoptotic cell death, induce inflammation, as well as inhibit tumorigenesis and viral replication. TNF-α and IL-1 commonly works simultaneously and synergistically in stimulating and sustaining inflammation within the body.

### General Discussion of Anti-Inflammatory Cytokines

Anti-inflammatory cytokines and anti-inflammatory mediators refer generally to immuno-regulatory cytokines that inhibit or counteract various aspects of inflammation. In other words, anti-inflammatory cytokines counteracts various biological effects of pro-inflammatory cytokines and pro-inflammatory mediators. Anti-inflammatory cytokines generally control or mitigate the magnitude of inflammation in vivo. Functions of anti-inflammatory cytokines include inhibiting production of pro-inflammatory cytokines and inhibiting cell activation.

Examples of anti-inflammatory cytokines include IL-2, IL-4, IL-10, and IL-13. IL-2 is a variably glycosylated single protein molecule having as mass of approximately 15.5 kD. IL-2 is generally produced by activated T helper cells (also known as effector T cells) during an immune response. Pathogens (also known as antigens) that invade or are introduced within the body bind to receptors that are found on the surfaces of lymphocytes. Binding of such pathogens or antigens to T cell receptors (TCR) stimulates secretion of IL-2. IL-2 mediates its effects by binding to IL-2 receptor molecules, which are expressed by lymphocytes. The binding of IL-2 to its receptor molecule triggers a signaling cascade, for example Ras/MAPK, JAK/Stat, and PI 3-kinase/Akt signaling modules.

The body produces IL-2 during immune responses. IL-2 has numerous functions including facilitating or effectuating production of immunoglobulins by B cells. In addition, IL-2 induces differentiation and proliferation of natural killer cells. IL-2 also causes a stimulation of growth, differentiation, and proliferation of antigen-selected cytotoxic T cells via an activation of expression of specific genes. IL-2 is considered to be important for the development of T cell immunologic memory. IL-2 is necessary during T cell development in the thymus for enabling the maturation of regulatory T cells, which are a unique subset of T cells.

### General Discussion of Viral Neuraminidase

A neuraminidase is a glycoside hydrolase enzyme that cleaves the glycosidic linkages of neuraminic acids. A viral neuraminidase is the neuraminidase enzyme present on the surface of a virus, for example an influenza virus.

Enzymatic activity of the viral neuraminidase enables the virus (e.g. the influenza virus) to be released from a host cell. The influenza virus's surface or membrane includes two glycoproteins, namely hemagglutinin and neuraminidase. The presence of hemagglutinin on the surface of the influenza virus inhibits the release of the influenza virus from the host cell. The viral neuraminidase cleaves terminal neuraminic acid residues (also known as sialic acid residues) from glycan structures on the surface of the host cell, thereby enabling the release of the influenza virus from the host cell.

Release of the influenza virus from the host cell causes the spread of the influenza virus to uninfected neighboring cells. In addition, the cleaving of neuraminic acid residues or sialic acid residues from proteins on the surfaces of the influenza viruses prevents an aggregation of the influenza viruses.

The viral neuraminidase is frequently an antigenic determinant for that virus. Accordingly, viral neuraminidase often serves as a target for anti-viral drugs. Drugs (e.g. pharmaceutical compositions) that inhibit the activity of viral neuraminidase have been suggested for use as anti-influenza drugs.

### General Discussion of Influenza A (H1N1)

Influenza, which is commonly referred to as the flu, is an infectious disease caused by RNA viruses of the family Orthomyxoviridae (i.e. influenza viruses). Influenza is typically transmitted through the air or contact with infected body fluids. Common symptoms of influenza include chills, fever, sore throat, muscle pains, headaches, coughing, and general discomfort.

Influenza A (H1N1) is a specific type of influenza that is caused by the influenza A (H1N1) virus. The influenza A (H1N1) virus is a subtype (i.e. a particular strain) of influenza virus A. Influenza A virus strains are typically categorized according to two proteins that are found on the surface of the influenza A virus, namely hemagglutinin (H) and neuraminidase (N). The structure, and number, of each of the two proteins differ between different influenza A virus strains. This difference is due to rapid genetic mutations of the influenza A viral genome. Each influenza virus A strain is assigned a H number and an N number based on the particular structure, form, or number of the H and N proteins on the surface of the influenza A virus.

The influenza A (H1N1) virus is approximately 80-120 nanometers in diameter and is generally spherical in shape. The influenza A (H1N1) virus includes a viral envelope. The viral envelope includes two main types of glycoproteins, which are wrapped about a central core. The central core includes a viral RNA genome and a number of viral proteins that package and protect the viral RNA genome. The viral RNA genome of the influenza A (H1N1) virus includes seven or eight pieces of segmented negative-sense RNA, wherein each piece of RNA includes either one or two genes.

Symptoms of influenza A (H1N1) are similar to that of a regular human flu, and include chills, fever, sore throat, muscle pains, headaches, coughing, and general discomfort. Influenza A (H1N1) has been classified as a global pandemic by the World Health Organization (WHO). The full extent of severity of Influenza A (H1N1) has not been ascertained. However, Influenza A (H1N1) is generally considered to be a significant threat to public health.

The following representative examples describe experiments showing particular effects of phytochemical compositions that include predetermined concentrations of at least one of xanthones and sesamin. While specific details associated with each experiment will be presented in the following examples, it will be understood by a person having ordinary skill in the art that the scope of the present disclosure is not limited to the following examples.

### Example One

Experiments were conducted to evaluate the effect of particular phytochemical compositions including particular concentrations of xanthones on quantity or level of IL-1 released from specified cells found within a body. The cells used for the experiment described in example one are lipopolysaccharide (LPS) induced peripheral blood mononuclear cells (PBMCs). It is understood by a person having ordinary skill in the art that the PBMCs may be substituted with other immune cells found within the body, for example lymphocytes, monocytes, and macrophages. It is also understood by a person having ordinary skill in the art that other inducing substances or chemicals besides LPS, for example viral antigens, may also be used for inducing the PBMCs.

To conduct the experiments of example one, PBMCs were isolated and prepared using methods or techniques known to a person having ordinary skill in the art. An exemplary method or technique is the differential density gradient centrifugation technique, the steps or process sequence of which are known to a person of ordinary skill in the art. In addition, LPS is obtained from a commercial source, for example Sigma-Aldrich Co.

The PBMCs are cultured in 10% fetal calf serum in RPMI1640 medium. Six cultures of PBMCs, i.e. culture 1 to culture 6, were prepared. The contents of each of cultures 1 to 6 were as follows:

(i) Culture 1: Control culture 1. Includes a predetermined volume of PBMCs (1×10⁵ PBMCs/culture);

(ii) Culture 2: Control culture 2. Includes a predetermined volume of PBMCs (1×10⁵ PBMCs/culture) and LPS at a concentration of 30 pg/ml;

(iii) Culture 3: Includes a predetermined volume of PBMCs (1×10⁵ PBMCs/culture), LPS at a concentration of 30 pg/ml, and a volume of a phytochemical composition providing a xanthone concentration of 0.001 ug/ml;

(iv) Culture 4: Includes a predetermined volume of PBMCs (1×10⁵ PBMCs/culture), LPS at a concentration of 30 pg/ml, and a volume of a phytochemical composition providing a xanthone concentration of 0.005 ug/ml;

(v) Culture 5: Includes a predetermined volume of PBMCs (1×10⁵ PBMCs/culture), LPS at a concentration of 30 pg/ml, and a volume of a phytochemical composition providing a xanthone concentration of 0.01 ug/ml; and

(vi) Culture 6: Includes a predetermined volume of PBMCs (1×10⁵ PBMCs/culture), LPS at a concentration of 30 pg/ml, and a volume of a phytochemical composition providing a xanthone concentration of 0.05 ug/ml.

In the experiments of example one, each of the cultures 1 to 6 is made up to a final volume of approximately 100 μl in a well of a standard 96 well plate. It will be understood by a person of ordinary skill in the art that alternative volumes may also be used. The number or concentration of PBMCs used for each culture is approximately equal. In the experiments of example 1, the number of PBMCs used for each culture is approximately 1×10⁵ (i.e., 100,000). It will be understood by a person of ordinary skill in the art that the number or concentration of PBMCs used for each of cultures 1 to 6 can be adjusted as required (e.g., to 150,000 or 200,000 PBMCs) by varying the degree of dilution of the cultures (e.g., by increasing total volume of the culture by adding medium).

Each of cultures 1 to 6 was incubated at the same physical for an equal length of time. More specifically, each culture was incubated at a temperature of 37° C. with 5% of carbon dioxide for 24 hours.

After a predetermined time period, the quantity of IL-1 in each of cultures 1 to 6 was quantified using the ELISA technique (e.g., Biotrak Easy ELISA Aersham). The quantity of IL-1 detected by the ELISA technique in each of cultures 1 to 6 reflects the quantity of IL-1 released by the PBMCs of that culture.

### Results

Results showed that LPS at a concentration of 30 pg/ml is capable of inducing IL-1 release from PBMCs. As seen in the control culture 2, addition of LPS at a concentration of 30 pg/ml induced an approximately 35% increase in IL-1 release from PBMCs. Results showed that cultures 4 to 6, which were incubated with phytochemical compositions that include xanthones of a concentration of at least 0.005 ug/ml, had a reduced level of IL-1 increase as compared to the control culture 2. Accordingly, results show that the quantity of IL-1 released from PBMCs is lower when the PBMCs are incubated with phytochemical compositions that include xanthones of a concentration of at least 0.005 ug/ml.

IL-1 released by PBMCs decreased by approximately 10% when the PBMCs were incubated with phytochemical compositions including 0.005 ug/ml of xanthones. IL-1 released by PBMCs decreased by approximately 15% when the PBMCs were incubated with phytochemical compositions including 0.01 ug/ml of xanthones. IL-1 released by PBMCs decreased by approximately 25% when the PBMCs were incubated with phytochemical compositions including 0.05 ug/ml of xanthones.

### Conclusion

The experiment of example one indicates that phytochemical compositions including predetermined concentrations of xanthones reduce an amount or quantity of IL-1 released by specified cells (e.g. PBMCs) found within a body of a living organism. The reduction of IL-1 released is between approximately 10% and approximately 25% with the phytochemical compositions of the concentrations stated above. Results showed that phytochemical compositions with higher concentrations of xanthones causes a greater decrease in the amount or quantity of IL-1 released by the cells found within the body. Accordingly, results indicate that increasing the concentration of xanthones can facilitate greater decreases in the quantity of IL-1 released by the cells of the body. Although the cells used for the experiment described in example two are PBMCs, a person having ordinary skill in the art will understand that the effects of the phytochemical compositions that include xanthones can be replicated or demonstrated with other immune cells found within the body.

### Example Two

Experiments were conducted to evaluate the effect of particular phytochemical compositions provided by the present disclosure on IL-2 gene expression relative to glyceraldehyde 3-phosphate dehydrogenase (GAPDH) gene expression in specified cells found within a body. The cells used for the experiments described in example two are LPS induced peripheral blood mononuclear cells (PBMCs). It is understood by a person having ordinary skill in the art that the PBMCs may be substituted with other immune cells found within the body. It is also understood by a person having ordinary skill in the art that other inducing substances or chemicals besides LPS, for example viral antigens, may also be used for inducing the PBMCs.

To conduct the experiments of example two, PBMCs were isolated and prepared using methods or techniques known to a person having ordinary skill in the art. An exemplary method or technique is the differential density gradient centrifugation technique, the steps or process steps of which are known to a person of ordinary skill in the art. In addition, LPS is obtained from a commercial source, for example Sigma-Aldrich Co.

The PBMCs are cultured in 10% fetal calf serum in RPMI1640 medium. Five cultures of PBMCs, i.e. culture 1 to culture 5, were prepared. The contents of cultures 1 to 5 were as follows:

(i) Culture 1: Control culture 1. Includes a predetermined volume of PBMCs (1×10⁵ PBMCs/culture);

(ii) Culture 2: Includes a predetermined volume of PBMCs (1×10⁵ PBMCs/culture) and a volume of phytochemical composition providing xanthones of a concentration of 0.001 ug/ml;

(iii) Culture 3: Includes a predetermined volume of PBMCs (1×10⁵ PBMCs/culture) and a volume of phytochemical composition providing xanthones of a concentration of 0.01 ug/ml;

(iv) Culture 4: Includes a predetermined volume of PBMCs (1×10⁵ PBMCs/culture) and a volume of phytochemical composition providing xanthones of a concentration of 0.1 ug/ml; and

(v) Culture 5: Includes a predetermined volume of PBMCs (1×10⁵ PBMCs/culture) and a volume of phytochemical composition providing xanthones of a concentration of 1.0 ug/ml.

In the experiments of example two, each of the cultures 1 to 5 is made up to a final volume of approximately 100 μl in a well of a standard 96 well plate. It will be understood by a person of ordinary skill in the art that alternative volumes may also be used. The number or concentration of PBMCs used for each culture is approximately equal. In the experiments of example two, the number of PBMCs used for each culture is approximately 1×10⁵ (i.e., 100,000). It will be understood by a person of ordinary skill in the art that the number or concentration of PBMCs used for each of cultures 1 to 5 can be adjusted as required (e.g., to 150,000 or 200,000 PBMCs) by varying the degree of dilution of the cultures (e.g., by increasing total volume of the culture by adding medium).

Each of cultures 1 to 5 was incubated at the same physical conditions for an equal length of time. More specifically, each culture was incubated at a temperature of 37° C. with 5% of carbon dioxide for 24 hours.

The amount or level of IL-2 gene expression in the PBMCs was measured or determined using methods or techniques known to a person having ordinary skill in the relevant art. In example two, Real-Time PCR (RT-PCR) was used for quantifying the amount of IL-2 gene expression in the PBMCs.

### Results

Results showed that PBMCs that were incubated with phytochemical compositions that include xanthones (i.e. PBMCs of cultures 2 to 5) displayed higher levels of IL-2 gene expression relative to GAPDH as compared to PMBCs of the control culture 1. More specifically, the level of IL-2 gene expression increased by between approximately 50% and approximately 70% when the PBMCs were incubated with phytochemical compositions including between 0.001 ug/ml and 1.0 ug/ml of xanthones (i.e., PBMCs of cultures 2 to 5) as compared to when PBMCs were not incubated with phytochemical compositions including xanthones (i.e., PBMCs of control culture 1).

### Conclusion

The experiments of example two indicate that phytochemical compositions including predetermined concentrations of xanthones increase the level of IL-2 gene expression of specified cells (e.g. PBMCs) found within a body of a living organism. The increase in IL-2 gene expression effected by phytochemical compositions including xanthones is between approximately 50% and approximately 70%. It will be understood by a person having ordinary skill in the art that the increase in IL-2 gene expression will result in a corresponding increase in release of IL-2 by PBMCs and hence an increase in IL-2 quantity within the body. Although the cells used for the experiments described in example two are PBMCs, a person having ordinary skill in the art will understand that the effects of the phytochemical compositions including xanthones can be replicated or demonstrated with other immune cells found within the body.

### Example Three

A computer graphic simulation and demonstration showing an inhibitory effect of xanthones against viral neuraminidase is also provided by the present disclosure. More specifically, the computer graphic simulation provided by example three shows the inhibitory effect of alpha-mangostin against neuraminidase of influenza A (H1N1) virus. An alpha-mangostin molecule binds to a specific site of the neuraminidase of the influenza A (H1N1) virus via the functional groups of the alpha-mangostin molecule. The computer simulation shows that a binding energy of −24.2 kcal/mol was associated with the binding of alpha-mangostin to the neuraminidase of the influenza A (H1N1) virus. The above binding energy indicates that the binding of alpha-mangostin with the neuraminidase of the influenza A (H1N1) virus facilitates or effectuates an inactivation or reduction in action or activity of the influenza A (H1N1) virus.

### Conclusion

The computer graphic simulation and demonstration provided by example three shows that xanthones (e.g. alpha-mangostin) include functional groups that are capable of binding to viral neuraminidase (e.g. neuraminidase of the influenza A (H1N1) virus). In addition, the binding energy of the xanthone molecule (e.g. alpha-mangostin molecule) and the viral neuraminidase (e.g. neuraminidase of the influenza A (H1N1) virus) indicates that the binding of xanthones (e.g. alpha-mangostin) to viral neuraminidase results in a more stable xanthone-viral neuraminidase compound relative to viral neuramnidase when considered alone. Accordingly, the binding of xanthones to the viral neuraminidase can inactivate or reduce the action or activity of the viral neuraminidase. The inactivation or reduction in action or activity of the viral neuraminidase confers upon xanthones, and phytochemical compositions including xanthones, anti-viral (e.g. anti-influenza A (H1N1) properties and uses.

### Example Four

A computer graphic simulation and demonstration showing inhibitory effect of sesamin against viral neuraminidase is also provided by the present disclosure. More specifically, the computer graphic simulation of this example shows the inhibitory effect of sesamin against neuraminidase of influenza A (H1N1) virus. Sesamin binds to specific sites of the neuraminidase of the influenza A (H1N1) virus via at least one functional group of the sesamin molecule. Computer simulation showed that a binding energy of −29.1 kcal/mol was associated with the specific binding between sesamin and the neuraminidase of the influenza A (H1N1) virus. The above binding energy of −29.1 kcal/mol indicates that the binding of sesamin with the neuraminidase of the influenza A (H1N1) virus facilitates or effectuates decrease or inactivation of action or activity of the influenza A (H1N1) virus.

### Conclusion

The computer graphic simulation and demonstration provided by example four shows that sesamin include at least one functional group that is capable of binding to viral neuraminidase (e.g. neuraminidase of the influenza A (H1N1) virus). In addition, the binding energy of the bond between the sesamin molecule and the viral neuraminidase indicates that the binding of sesamin to viral neuraminidase results in a more stable sesamin-viral neuraminidase compound relative the viral neuraminidase when considered alone. Accordingly, the binding energy indicates that the binding of the sesamin molecule and the viral neuraminidase can inactivate or reduce the action or activity of the viral neuraminidase. The inactivation or reduction in action or activity of the viral neuraminidase confers upon sesamin, and phytochemical compositions including sesamin, an anti-viral (e.g. anti-influenza A (H1N1) properties or use.

### Example Five

Experiments were conducted to evaluate the effect of particular phytochemical compositions provided by the present disclosure on IL-1 gene expression in specified cells found within a body of a living organism. The cells used for the experiment described in example one are lipopolysaccharide (LPS) induced synovial fibroblast (SF) cells. The SF cells were isolated from synovium tissue (also referred to as synovial tissue) using a standard cell isolation technique or method that is known to a person of ordinary skill in the art. In addition, LPS is obtained from a commercial source, for example Sigma-Aldrich Co. It is understood by a person having ordinary skill in the art that the SF cells may be substituted with other immune cells found within the body, for example lymphocytes, monocytes, and macrophages. It is also understood by a person having ordinary skill in the art that other inducing substances or chemicals besides LPS, for example viral antigens, may also be used for inducing the SF cells.

Nine cultures of SF cells, i.e. culture 1 to culture 9, were prepared. The contents of the each of cultures 1 to 9 were as follows:

(i) Culture 1: First control culture. Includes a predetermined volume of SF cells.

(ii) Culture 2: Second control culture. Includes the predetermined volume of SF cells induced with 0.2 μg/ml of LPS.

(iii) Culture 3: Third control culture. Includes the predetermined volume of SF cells induced with 0.2 μg/ml of LPS, and 5 μl of dimethyl sulfoxide (DMSO);

(iv) Culture 4: Includes the predetermined volume of SF cells induced with 0.2 μg/ml of LPS, and a phytochemical composition providing 0.025 mM of sesamin;

(v) Culture 5: Includes the predetermined volume of SF cells induced with 0.2 μg/ml of LPS, and a phytochemical composition providing 0.05 mM of sesamin;

(vi) Culture 6: Includes the predetermined volume of SF cells induced with 0.2 μg/ml of LPS, and a phytochemical composition providing 0.10 mM of sesamin;

(vii) Culture 7: Includes the predetermined volume of SF cells induced with 0.2 μg/ml of LPS, and a phytochemical composition providing 0.25 mM of sesamin;

(viii) Culture 8: Includes the predetermined volume of SF cells induced with 0.2 μg/ml of LPS, and a phytochemical composition providing 0.50 mM of sesamin; and

(ix) Culture 9: Includes the predetermined volume of SF cells induced with 0.2 μg/ml of LPS, and a phytochemical composition providing 1.0 mM of sesamin.

In the experiments of example five, approximately 5.0 ml of SF cells were used for each culture. It will be understood by a person of ordinary skill in the art that alternative volumes of SF cells may also be used. The SF cells were cultured in T-25 flasks. The number or concentration of SF cells used in each of cultures 1 to 9 is approximately equal. For the experiments of example 1, approximately 1×10⁵ (i.e., 100,000) SF cells were present in each culture. It will be understood by a person of ordinary skill in the art that the number or concentration of SF cells used in each of cultures 1 to 9 can be adjusted as required by varying the degree of dilution of the cultures (e.g., by increasing total volume of the culture by adding medium).

Each of cultures 1 to 9 was incubated at the same physical conditions (e.g., same temperate, oxygen concentration, and amount of agitation) for an equal length of time, for instance 24 hours.

The amount or level of IL-1 gene expression in the SF cells was measured or determined using methods or techniques known to a person having ordinary skill in the relevant art. In example five, Real-Time PCR (RT-PCR) was used for quantifying the amount of IL-1 gene expression in the SF cells. IL-1beta or IL-1β specific primers were used during RT-PCR, thereby facilitating or effectuating the quantification of IL-1β gene expression in the SF cells. However, it will be understood by a person having ordinary skill in the relevant art that IL-1β gene expression directly mirrors IL-1βα gene expression, and therefore the overall IL-1 gene expression in the SF cells.

### Results

The IL-1 gene expression relative to glyceraldehydes-3-phosphate dehydrogenase (GAPDH) gene expression of SF cells in each of cultures 1 to 9 was measured using RT-PCR. Results show that the IL-1 gene expression relative to GAPDH was lower in SF cells of cultures that were incubated with phytochemical compositions that include predetermined concentrations of sesamin. The IL-1 gene expression relative to GAPDH decreased by approximately 20% and approximately 65% in cultures 4 to 9, i.e. in cultures that were incubated with phytochemical compositions that include predetermined concentrations of sesamin, as compared to either one of control cultures 1 to 3. The maximal decrease in gene expression is seen with culture 9, which comprises the phytochemical composition having sesamin concentration of 1.0 mM. The IL-1 gene expression relative to GAPDH decreased by approximately 65% in culture 9 as compared to control culture 2. The minimal decrease in gene expression is seen with culture 4, which comprises the phytochemical composition having sesamin concentration of 0.025 mM. The IL-1 gene expression relative to GAPDH decreased by approximately 20% in culture 4 as compared to control culture 3. Results indicate that cultures incubated with phytochemical compositions having higher concentrations of sesamin produce a correspondingly greater decrease in IL-1 gene expression relative to GAPDH in SF cells.

### Conclusion

The experiment of example five shows that phytochemical compositions that include sesamin are able to reduce IL-1 gene expression in cells found within the body (e.g., synovial fibroblast cells). More specifically, phytochemical compositions that include sesamin are able to reduce IL-1 gene expression in specific cells of the body by between approximately 20% and approximately 65%. In addition, phytochemical compositions with higher concentrations of sesamin are able to produce a greater decrease in IL-1 gene expression in cells of the body. Although the cells used for the experiment described in example one are SF cells, a person of ordinary skill in the art will understand that the effects of the phytochemical compositions that include sesamin can be replicated or demonstrated with other immune cells found within the body.

### Example Six

Experiments were conducted to evaluate the effect of particular phytochemical compositions provided by the present disclosure on quantity of IL-1 released by specified cells found within a body of a living organism. Cells used for the experiment described in this example are LPS induced peripheral blood mononuclear cells (PBMCs). It is understood by a person having ordinary skill in the art that the PBMCs may be substituted for other immune cells found within the body. It is also understood by a person having ordinary skill in the art that other inducing substances or chemicals besides LPS, for example viral antigens, may also be used for inducing the PBMCs.

To conduct the experiments of example six, PBMCs were isolated and prepared using methods or techniques known to a person having ordinary skill in the art. An exemplary method or techniques is the differential density gradient centrifugation technique, the steps or process steps of which is known to a person of ordinary skill in the art. In addition, LPS can be obtained from a commercial source, for example Sigma-Aldrich Co.

The PBMCs are cultured in 10% fetal calf serum in RPMI1640 medium. Seven cultures of PBMCs, i.e. culture 1 to culture 7, were prepared. The contents of each of cultures 1 to 7 were as follows:

(i) Culture 1: First control culture. Includes a predetermined volume of PBMCs (1×10⁵ PBMCs/culture);

(ii) Culture 2: Second control culture. Includes the predetermined volume of PBMCs (1×10⁵ PBMCs/culture) and LPS at a concentration of 20 ng/ml;

(iii) Culture 3: Includes the predetermined volume of the specified concentration of PBMCs, LPS at a concentration of 20 ng/ml, and a volume of phytochemical composition providing 0.1 uM of sesamin;

(iv) Culture 4: Includes the predetermined volume of PBMCs (1×10⁵ PBMCs/culture), LPS at a concentration of 20 ng/ml, and a volume of phytochemical composition providing 0.5 uM of sesamin;

(v) Culture 5: Includes the predetermined volume of PBMCs (1×10⁵ PBMCs/culture), LPS at a concentration of 20 ng/ml, and a volume of phytochemical composition providing 1.0 uM of sesamin;

(vi) Culture 6: Includes the predetermined volume of PBMCs (1×10⁵ PBMCs/culture), LPS at a concentration of 20 ng/ml, and a volume of phytochemical composition providing 5.0 uM of sesamin; and

(vii) Culture 7: Includes the predetermined volume of PBMCs (1×10⁵ PBMCs/culture), LPS at a concentration of 20 ng/ml, and a volume of phytochemical composition providing 10 uM of sesamin.

In the experiments of example six, each of the cultures 1 to 7 is made up to a final volume of approximately 100 μl in a well of a standard 96 well plate. It will be understood by a person of ordinary skill in the art that alternative volumes may also be used. The number or concentration of PBMCs used for each culture is approximately equal. For instance, the number of PBMCs used for each culture is approximately 1×10⁵ (i.e., 100,000). It will be understood by a person of ordinary skill in the art that the number or concentration of PBMCs used for each of cultures 1 to 7 can be adjusted as required by varying the degree of dilution of the cultures (e.g., by increasing total volume of the culture by adding medium).

Each culture was incubated at the same physical conditions for an equal length of time. More specifically, each culture was incubated at a temperature of 37° C. with 5% of carbon dioxide for 24 hours.

After a predetermined time period, quantity of IL-1 in each of cultures 1 to 7 was quantified using the ELISA technique (e.g., Biotrak Easy ELISA Aersham). The quantity of IL-1 detected by the ELISA technique in each of cultures 1 to 7 reflects the quantity of IL-1 released by the PBMCs of that culture.

### Results

Results showed that the quantity or level of IL-1 (pg/ml) was lower in cultures that were incubated with phytochemical compositions that included sesamin. The quantity of IL-1 (pg/ml) decreased by between approximately 5% and approximately 35% in cultures that were incubated with phytochemical compositions that include sesamin (i.e., in cultures 3 to 7). The minimum decrease was observed with cultures 3 and 4, which showed an approximately 5% decrease in quantity of IL-1 as compared to control culture 1. The maximum decrease was observed with culture 7, which showed an approximately 35% decrease in quantity of IL-1 as compared to control culture 2. Results indicate that the quantity of IL-1 decreased by a greater amount in cultures that were incubated with phytochemical compositions having higher concentrations of sesamin.

### Conclusion

The experiments of example six indicate that phytochemical compositions that include sesamin are able to reduce the amount or quantity of IL-1 released by specified cells (e.g. PBMCs) found within a body of a living organism. The reduction of quantity of IL-1 released is between approximately 5% and approximately 35%. In addition, phytochemical compositions having higher concentrations of sesamin are able to produce or effect a greater decrease in the amount or quantity of IL-1 released by the cells found within the body. Although the cells used for the experiments described in example two are PBMCs, a person having ordinary skill in the art will understand that the effects of the phytochemical compositions having sesamin can be replicated or demonstrated with other immune cells found within the body.

### Example Seven

Experiments were conducted to evaluate the effect of particular phytochemical compositions provided by the present disclosure on IL-2 gene expression in specified cells found within a body of a living organism. Cells used for the experiment described in this example are LPS induced peripheral blood mononuclear cells (PBMCs). It is understood by a person having ordinary skill in the art that the PBMCs may be substituted for other immune cells found within the body. It is also understood by a person having ordinary skill in the art that other inducing substances or chemicals besides LPS, for example viral antigens, may also be used for inducing the PBMCs.

To conduct the experiments of example seven, PBMCs were isolated and prepared using methods or techniques known to a person having ordinary skill in the art. An exemplary method or techniques is the differential density gradient centrifugation technique, the steps or process steps of which is known to a person of ordinary skill in the art. In addition, LPS can be obtained from a commercial source, for example Sigma-Aldrich Co.

The PBMCs are cultured in 10% fetal calf serum in RPMI1640 medium. Five cultures of PBMCs, i.e. culture 1 to culture 5, were prepared. The contents of each of cultures 1 to 5 were as follows:

Culture 1: Control culture. Includes a predetermined volume of PBMCs (1×10⁵ PBMCs/culture);

(ii) Culture 2: Includes the predetermined volume of PBMCs (1×10⁵ PBMCs/culture), LPS at a concentration of 20 ng/ml, and a volume of phytochemical composition providing 0.1 ug/ml of sesamin;

(iii) Culture 3: Includes the predetermined volume of PBMCs (1×10⁵ PBMCs/culture), LPS at a concentration of 20 ng/ml, and a volume of phytochemical composition providing 0.5 ug/ml of sesamin;

(iv) Culture 4: Includes the predetermined volume of PBMCs (1×10⁵ PBMCs/culture), LPS at a concentration of 20 ng/ml, and a volume of phytochemical composition providing 1.0 ug/ml of sesamin; and

Culture 5: Includes the predetermined volume of of PBMCs (1×10⁵ PBMCs/culture), LPS at a concentration of 20 ng/ml, and a volume of phytochemical composition providing 5.0 ug/ml of sesamin.

In the experiments of example seven, each of the cultures 1 to 5 is made up to a final volume of approximately 100 μl in standard 96 well plates. It will be understood by a person of ordinary skill in the art that alternative volumes may also be used. The number or concentration of PBMCs used for each culture is approximately equal. For instance, the number of PBMCs used for each culture is approximately 1×10⁵ (i.e., 100,000). It will be understood by a person of ordinary skill in the art that the number or concentration of PBMCs used for each of cultures 1 to 5 can be adjusted as required by varying the degree of dilution of the cultures (e.g., by increasing total volume of the culture by adding medium).

Each of cultures 1 to 5 was incubated at the same physical conditions for an equal length of time. More specifically, each culture was incubated at a temperature of 37° C. with 5% of carbon dioxide for 24 hours.

The amount or level of IL-2 gene expression in the PBMCs was measured or determined using methods or techniques known to a person having ordinary skill in the relevant art. In example three, Real-Time PCR (RT-PCR) was used for quantifying the amount of IL-2 gene expression in the PBMCs.

### Results

Results showed that the level of IL-2 gene expression relative to GAPDH increases in PBMCs of cultures that were incubated with phytochemical compositions having predetermined concentrations of sesamin (i.e. cultures 2 to 5). In addition, the results showed that phytochemical compositions including predetermined concentrations of sesamin produces an increase in IL-2 gene expression relative GAPDH of between approximately 40% and approximately 220%. The increase in IL-2 gene expression relative GAPDH is greatest, more specifically approximately 220%, in PBMCs that were incubated with the phytochemical composition having 1.0 ug/ml of sesamin (i.e. in culture 4). Results indicate that the increase in IL-2 gene expression in PBMCs increases when the PBMCs are incubated with phytochemical compositions of increasing sesamin concentration, more specifically from 0.1 ug/ml to 1.0 ug/ml. Results suggest that maximal increase in IL-2 gene expression is achieved with phytochemical compositions of sesamin concentrations less than 5.0 ug/ml.

### Conclusion

The experiment of example seven shows that phytochemical compositions that include sesamin increase IL-2 gene expression in specific cells found within the body (e.g. PBMCs). More specifically, phytochemical compositions that include sesamin increase IL-2 gene expression in specific cells of the body by between approximately 40% and approximately 220%. Phytochemical compositions having increasing concentrations of sesamin from 0.1 ug/ml to 1.0 ug/ml produce the increasing increase in IL-2 gene expression in the cells of the body. However, maximal increase in IL-2 gene expression in the cells of the body is achieved with phytochemical compositions having sesamin concentrations of less than 5.0 ug/ml. Although the cells used for the experiment described in example three are PBMCs, a person having ordinary skill in the art will understand that the effects of the phytochemical compositions including sesamin can be replicated or demonstrated with other immune cells found within the body.

### Example Eight

Experiments were conducted to evaluate the effects of particular phytochemical compositions provided by the present disclosure on quantity of IL-1 released by viral-infected cells. Viral-infected cells used for the experiment described in this example are influenza A (H1N1) virus-infected cells. More specifically, the virus-Infected cells used for the experiments described in this example are influenza A (H1N1) virus-infected egg cells. The influenza A (H1N1) virus can be isolated from patients infected with the influenza A (H1N1) using standard techniques known to a person of ordinary skill in the art. In addition, isolation of cells (e.g., egg cells), and the infection of cells (e.g., isolated egg cells) can be performed using standard techniques or method known to a person of ordinary skill in the art. It is understood by a person having ordinary skill in the art that the influenza A (H1N1) virus-infected egg cells may be substituted for other viral-infected cells or other cells within the body, for instance, cells in which inflammation is stimulated or occurs.

Five cultures of cells, i.e., culture 1 to culture 5, were prepared. The contents of each of cultures 1 to 5 are as follows:

(ii) Culture 1: Control culture 1. Includes a predetermined volume of a sample of isolated egg cells that has been diluted in a ratio of 1:50;

(iii) Culture 2: Control culture 2. Includes a predetermined volume of a sample of isolated egg cells that has been diluted in a ratio of 1:50 similar to that of culture 1 that have been infected with the influenza A (H1N1) virus diluted at a ratio of 1:7812500;

(iv) Culture 3: Control culture 2. Includes a predetermined volume of a sample of isolated egg cells that has been diluted in a ratio of 1:50 similar to that of culture 1 and DMSO that have been infected with the influenza A (H1N1) virus diluted at a ratio of 1:7812500;

(v) Culture 4: Includes the predetermined volume of the sample of isolated egg cells that have been infected with the influenza A (H1N1) virus similar to that of culture 2, as well as a volume of phytochemical composition providing xanthones at a concentration of 0.5 ug/ml; and

(vi) Culture 5: Includes the predetermined volume of the sample of isolated egg cells that have been infected with the influenza A (H1N1) virus similar to that of culture 2, as well as a volume of phytochemical composition providing xanthones at a concentration of 1.0 ug/ml.

In the experiments of example eight, each of cultures 1 to 5 is made up to a final volume of approximately 200 μl in a well of a standard 96 well plate. It will be understood by a person of ordinary skill in the art that alternative volumes (e.g., 100 μl) may also be used. The number or concentration of isolated egg cells used for each culture is approximately equal. For instance, the number of isolated used for each culture is approximately 2×10⁵ (i.e., 200,000). It will be understood by a person of ordinary skill in the art that the number or concentration of isolated egg cells used for each of cultures 1 to 5 can be adjusted as required by varying the degree of dilution of the cultures (e.g., by increasing total volume of the culture by adding medium).

Each of cultures 1 to 5 was incubated at the same physical conditions for an equal length of time. More specifically, each culture was incubated at a temperature of 37° C. with 5% of carbon dioxide for 24 hours. The amount of IL-1 released from the cells were measured or determined using methods or techniques known to a person having ordinary skill in the relevant art.

### Results

Results show that the quantity of IL-1 released from cells that are infected with the influenza A (H1N1) virus (i.e., cells of control cultures 2 and 3) is higher than the quantity of IL-1 released from cells that are not infected with the influenza A (H1N1) virus (i.e., cells of control culture 1). More specifically, approximately between 28 pg/ml and 31 pg/ml of IL-1 was released by influenza A (H1N1) virus-infected cells (i.e., cells of control cultures 2 and 3) as compared to approximately 19 pg/ml of IL-1 that was released by cells that were not viral-infected (i.e., cells of control culture 1). This represents an increase in IL-1 released by approximately between 9 pg/ml and 12 pg/ml. In other words, the quantity of IL-1 released by influenza A (H1N1) virus-infected cells (i.e., cells of control cultures 2 and 3) increased by between approximately 45% and 65% as compared to the quantity of IL-1 released by cells that were not viral infected (i.e., cells of control culture 1). Such an increase in quantity of IL-1 released corresponds to a significant immune response in the body of a living organism.

Results show that the quantity of IL-1 released from the influenza A (H1N1) virus-infected cells is lower in cultures that further include phytochemical compositions that have predetermined concentrations of xanthones (i.e., 0.5 ug/ml and 1.0 ug/ml).

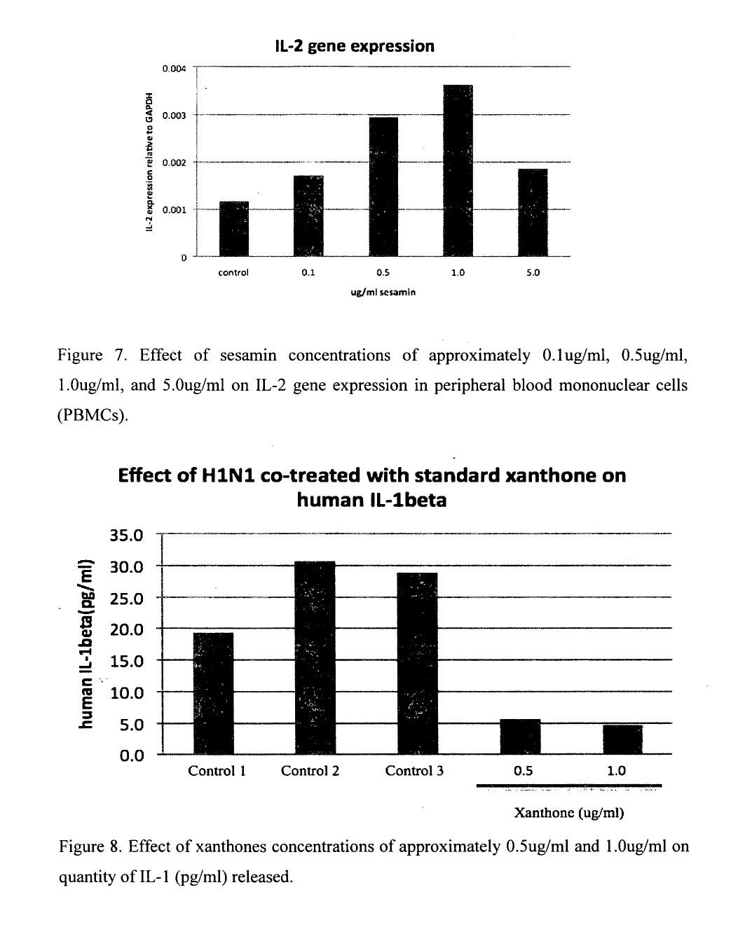

As shown in FIG. 8, the quantity of IL-1 released by influenza A (H1N1) virus-infected cells incubated with phytochemical compositions including 0.5 ug/ml of xanthones (i.e., in culture 4) was approximately 6 pg/ml. This represents between approximately 22 pg/ml and 25 pg/ml decrease in the quantity of IL-1 released by influenza A (H1N1) virus-infected cells when said cells are incubated with phytochemical compositions including 0.5 ug/ml of xanthones (i.e., in culture 4) as compared to when said cells were not incubated with any amount of xanthones (i.e., in control cultures 2 and 3).

As also shown in FIG. 8, the quantity of IL-1 released by influenza A (H1N1) virus-infected cells incubated with phytochemical compositions including 1.0 ug/ml of xanthones (i.e., in culture 5) was approximately 4.5 pg/ml. This represents between approximately 23.5 pg/ml and 26.5 pg/ml decrease in the quantity of IL-1 released by influenza A (H1N1) virus-infected cells when said cells were incubated with phytochemical compositions including 1.0 ug/ml of xanthones (i.e., in culture 5) as compared to when said cells were not incubated with any amount of sesamin (i.e., in control cultures 2 and 3).

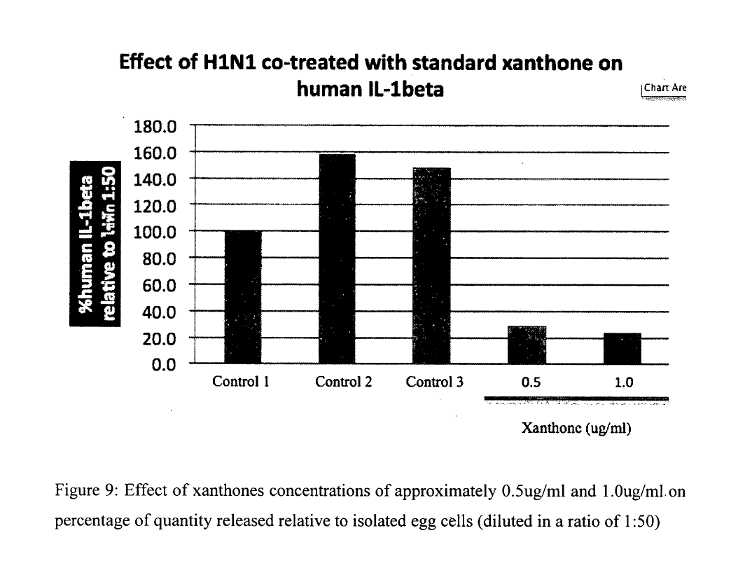

As shown in FIG. 9, the percentage of quantity of human IL-1 relative the quantity of isolated egg cells (diluted in a ratio of 1:50) was determined for each of cultures 1 to 5.

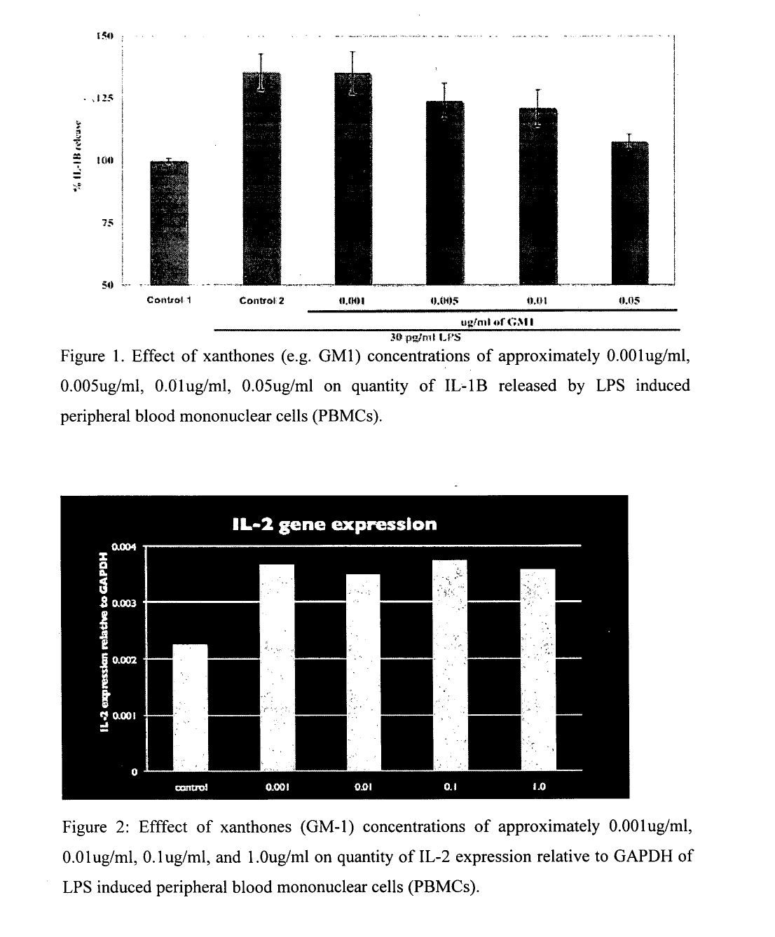

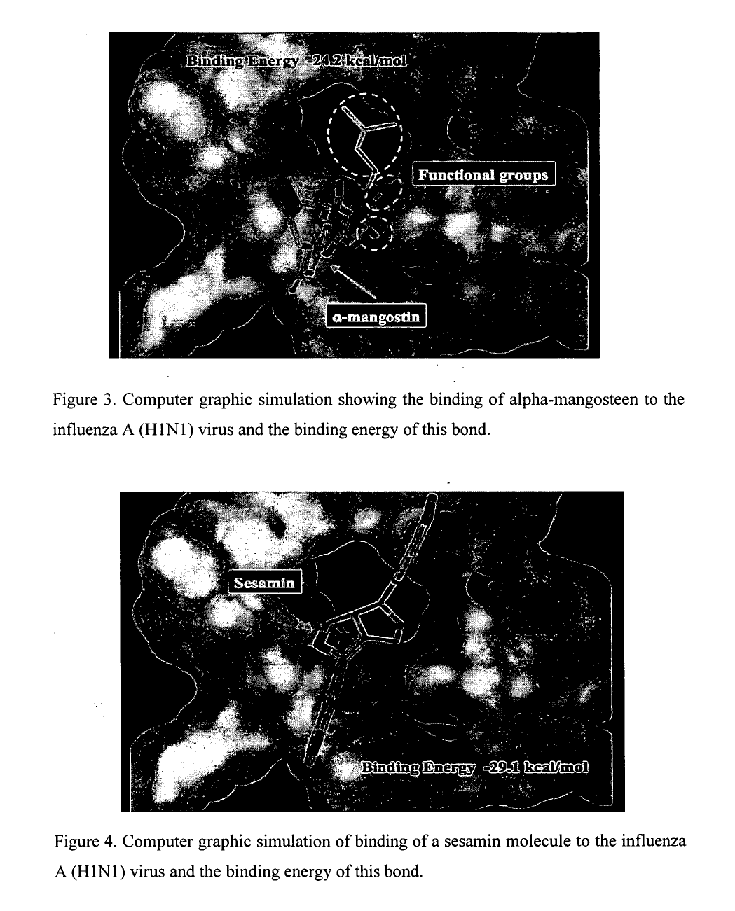

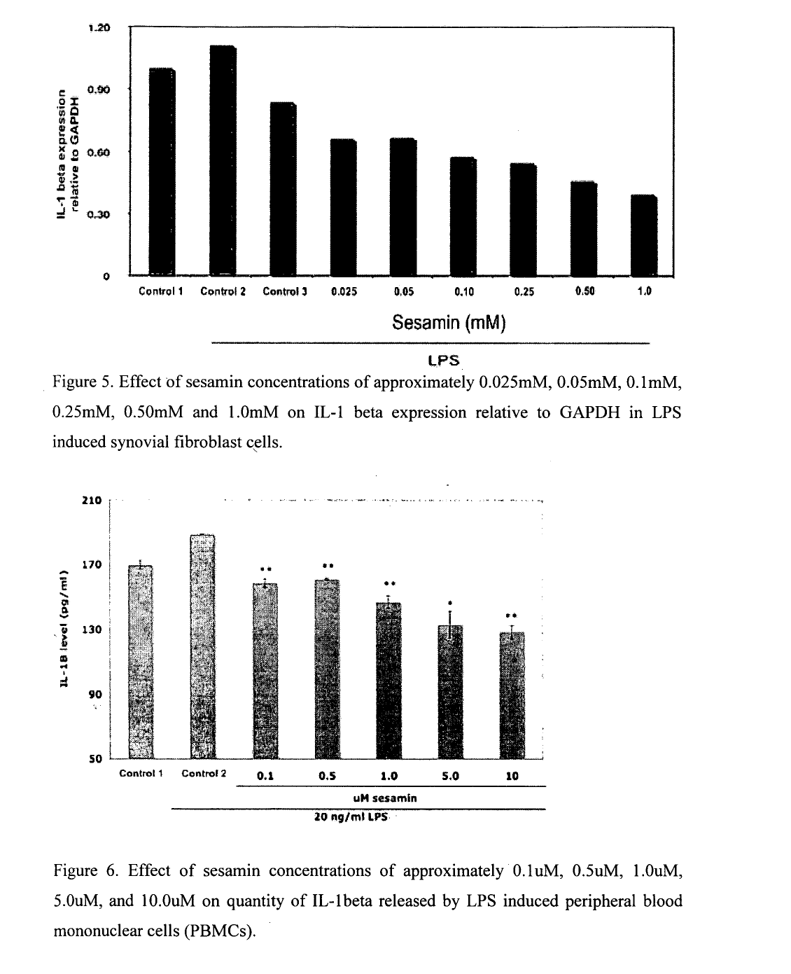

Results show that the percentage of quantity of human IL-1 relative the quantity of isolated egg cells (diluted in a ratio of 1:50) increased when the isolated egg cells were infected with the influenza A (H1N1) virus diluted at a ratio of 1:7812500. As shown in FIG. 6, the percentage of quantity of human IL-1 relative the quantity of isolated egg cells (diluted in a ratio of 1:50) increased from 100% to between approximately 145% and 160% when the isolated egg cells were infected with the influenza A (H1N1) virus diluted at a ratio of 1:7812500 (i.e., in cultures 2 and 3) as compared to when said cells were not infected with the influenza A (H1N1) virus diluted at a ratio of 1:7812500 (i.e., in culture 1).

Results also show that the percentage of quantity of human IL-1 relative the quantity of isolated egg cells (diluted in a ratio of 1:50) decreased when the cells that were infected with the influenza A (H1N1) virus diluted at a ratio of 1:7812500 were incubated with the phytochemical compositions including predetermined concentrations of xanthones (e.g., 0.5 ug/ml and 1.0 ug/ml) as compared to when said cells were not incubated with any concentration of xanthones.

As shown in FIG. 9, the percentage of quantity of human IL-1 relative to quantity of isolated egg cells diluted in a ration of 1:50 was between approximately 25% and approximately 30% when isolated eggs cells that were infected with the influenza A (H1N1) virus diluted at a ratio of 1:7812500 were incubated with the phytochemical composition including 0.5 ug/ml of xanthones. This represents between an approximately 115% and an approximately 135% decrease in the quantity of human IL-1 released relative to quantity of isolated egg cells diluted in a ration of 1:50) when the isolated eggs cells that were infected with the influenza A (H1N1) virus diluted at a ratio of 1:7812500 were incubated with the phytochemical composition including 0.5 ug/ml of xanthones (i.e., in culture 4) as compared to when the said cells were not incubated with any concentration of xanthones (i.e., in cultures 2 and 3).

As also shown in FIG. 9, the percentage of quantity of human IL-1 released relative to quantity of isolated egg cells diluted in a ration of 1:50) was between approximately 20% and approximately 25% when isolated eggs cells that were infected with the influenza A (H1N1) virus diluted at a ratio of 1:7812500 were incubated with the phytochemical composition including 1.0 ug/ml of sesamin. This represents between an approximately 120% and an approximately 140% decrease in the quantity of human IL-1 released relative to quantity of isolated egg cells diluted in a ration of 1:50) when the isolated eggs cells that were infected with the influenza A (H1N1) virus diluted at a ratio of 1:7812500 were incubated with the phytochemical composition including 1.0 ug/ml of xanthones (i.e., in culture 5) as compared to when the said cells were not incubated with any concentration of xanthones (i.e., in cultures 2 and 3).

### Conclusion

The experiments of example eight show that phytochemical compositions that include predetermined concentrations of xanthones are able to facilitate or cause a decrease in a quantity in IL-1 released by Influenza A (H1N1) virus-infected cells. More specifically, phytochemical compositions including xanthones concentration of between approximately 0.5 ug/ml and 1.0 ug/ml facilitate or cause a decrease in IL-1 released by Influenza A (H1N1) virus-infected cells of approximately between approximately 115% and 140%. Although the cells used for the, experiment described in example eight are Influenza A (H1N1) virus-infected egg cells, a person having ordinary skill in the art will understand that the effects of the phytochemical compositions including xanthones as shown in this example can be replicated or demonstrated with other viral infected cells, or other cells that are involved in viral infection or inflammatory processes within the body.

The amount or percentage decrease in quantity of IL-1 released by Influenza A (H1N1) virus-infected cells produced or facilitated by particular phytochemical composition including xanthones as shown in example eight is significantly better than expected (i.e., surprisingly better) to a person having ordinary skill in the art.

Embodiments of the present disclosure provide phytochemical compositions that include predetermined concentrations of xanthones for therapeutically affecting a pro-inflammatory cytokine condition and an anti-inflammatory cytokine condition in living organisms. In several embodiments, particular phytochemical compositions facilitates or effectuates at least one of reduction in quantity of a pro-inflammatory cytokine, increase in quantity of an anti-inflammatory cytokine, and inhibition of action of a viral neuraminidase (e.g., influenza A viral neuraminidase) within the body of the living organism.

Some embodiments of the present disclosure provide phytochemical compositions that include predetermined concentrations of both xanthones and sesamin for therapeutically affecting a pro-inflammatory cytokine condition and an anti-inflammatory cytokine condition in living organisms, for instance facilitating or effectuating at least one of reduction in quantity of a pro-inflammatory cytokine, increase in quantity of an anti-inflammatory cytokine, and inhibition of action of a viral neuraminidase (e.g., influenza A viral neuraminidase) within the body of the living organism.

The phytochemical composition provided by many embodiments of the present disclosure provides anti-inflammatory effects or uses. In addition, the phytochemical composition provided by some embodiments of the present disclosure provides anti-viral (e.g., anti-influenza) effects or uses (e.g., a viral activity inhibitory effect). In many embodiments, the reduction of the quantity of pro-inflammatory cytokines within the body of the living organism facilitates or effectuates prevention, control, down-regulation, and/or termination of the occurrence of cytokine storms or un-regulated inflammation within the body of the living organism. In several embodiments, phytochemical compositions provided by the present disclosure facilitates or effectuates at least one of prevention, control, down-regulation, or termination of cytokine storms or inflammation caused or triggered by viral infections (e.g., infections by influenza viruses such as influenza A (H1N1), influenza A (H1N2), influenza A (H2N1), influenza A (H2N2), and influenza A (H2N3), and influenza A (H5N1) viruses).

In specific embodiments, particular phytochemical compositions provided by the present disclosure are effective for reducing or beneficially modulating physical effects (e.g., tissue damage) caused at least partly by occurrences of cytokine storms within the body. In specific embodiments, the phytochemical compositions can be applied for prevention, treatment, or control of various infectious and non-infectious diseases. In selected embodiments, particular phytochemical compositions provided by the present disclosure are useful or effective against inflammation produced during infectious and non-infectious diseases (e.g., Osteoarthritis, Osteoporosis).

Although the representative examples provided above describes particular effects of xanthones and sesamin when taken singularly, phytochemical compositions in accordance with the present disclosure that include predetermined concentrations of both xanthones and sesamin can provide a greater decrease in the quantity of pro-inflammatory cytokines, a greater increase in the quantity of anti-inflammatory cytokines, and an enhanced inhibition of the action of the viral neuraminidase within the body of the living organism as compared to phytochemical compositions that include predetermined concentrations of either one of xanthones or sesamin alone. In most embodiments, the anti-inflammatory effect and/or anti-viral (e.g., anti-influenza) effect of the phytochemical composition is enhanced where the phytochemical composition includes predetermined concentrations of both xanthones and sesamin as compared to where the phytochemical composition includes predetermined concentration of either one of xanthones or sesamin. In many embodiments of the present disclosure, the above-described effect of the combination of xanthones and sesamin is synergistic in nature, giving rise to an unexpected or surprising level of benefit.

In addition, embodiments of the present disclosure also provide uses of xanthones for manufacture of particular phytochemical compositions provided by the present disclosure. In addition, some embodiments of the present disclosure provide uses of xanthones in combination with sesamin for manufacture of particular phytochemical compositions provided by the present disclosure. In some embodiments, the manufactured phytochemical composition is, or forms a portion of a food product (e.g., is manufactured primarily as a food product). In other embodiments, the phytochemical composition is, or forms a portion of a beverage product (e.g., is manufactured primarily as a beverage product). In other embodiments, the phytochemical composition is, or forms a portion of, a drug or pharmaceutical product. In other embodiments, the phytochemical composition is a supplement, additive, or ingredient for one of a food product, a beverage product, and a drug, pharmaceutical, or other consumable (e.g., ingestible) product. In many embodiments, the phytochemical composition is in the form of one of a tablet, a pellet, a powder, or an emulsion. In some embodiments, particular phytochemical compositions provided by the present disclosure are combined, mixed, synthesized, or manufactured with drugs, pharmaceutical compositions, phytochemical compositions, and/or nutraceutical compositions.

Although embodiments and representative examples and experiments of the present disclosure are described above, the present invention is not to be limited to specific details so described. The scope of the present disclosure is not limited to the embodiments of the present disclosure provided above. A person having ordinary skill in the art will understand that numerous changes and modifications can be made to the embodiments, examples, and experiments without departing from the scope or spirit of the present disclosure.

1. A composition comprising a concentration of xanthones of substantially between approximately 0.001 ug/ml and approximately 1.0 ug/ml, the composition for at least one of facilitating and effectuating decrease in quantity of a pro-inflammatory cytokine within a living organism when consumed thereby.

2. The composition as in claim 1, wherein the concentration of xanthones is substantially between approximately 0.005 ug/ml and approximately 0.5 ug/ml.

3. (canceled)

4. The composition as in claim 1, wherein the decrease in quantity of the pro-inflammatory cytokine within the living organism is substantially between approximately 10% and approximately 50%.

5. (canceled)

6. The composition as in claim 1, wherein the composition further at least one of facilitates and effectuates decrease in gene expression of the pro-inflammatory cytokine within living organism when consumed thereby.

7. The composition as in claim 1, wherein the pro-inflammatory cytokine is one of interleukin-1 and tumor necrosis factor-alpha.

8. The composition as in claim 1, wherein the composition at least one of facilitates and effectuates at least one of prevention, control, and termination of inflammation within the living organism.

9. The composition as in claim 1, wherein the composition further at least one of facilitates and effectuates an increase in quantity of one of an anti-inflammatory cytokine and an anti-inflammatory mediator within the living organism when consumed thereby.

10. The composition as in claim 9, wherein the composition at least one of facilitates and effectuates an increase in gene expression of the one of the anti-inflammatory cytokine and the anti-inflammatory mediator within the living organism when consumed thereby.

11. The composition as in claim 10, wherein the increase in gene expression of the one of the anti-inflammatory cytokine and the anti-inflammatory mediator is substantially between approximately 25% and approximately 100%.

12. (canceled)

13. The composition as in claim 11, wherein the one of the anti-inflammatory cytokine and the anti-inflammatory mediator is interleukin-2.

14. The composition as in claim 1, wherein the composition further at least one of facilitates and effectuates inhibition of a viral neuraminidase to thereby provide a viral activity inhibitory effect within the living organism.

15. The composition as in claim 14, wherein the viral neuraminidase is a neuraminidase of an influenza virus, the influenza virus being one of influenza A (H1N1), influenza A (H1N2), influenza A (H2N1), influenza A (H2N2), influenza A (H3N2), and influenza A (H5N1) virus, the inhibition of the neuraminidase of the influenza virus providing an anti-influenza effect within the living organism.

16. The composition as in claim 1, wherein the composition is one of a food, a beverage, a drug, and one of a supplement and additive substance for one of the food, the beverage, and the drug.

17. The composition as in claim 1, further comprising at least one of an anti-inflammatory substance, a connective tissue anti-degradation substance, an anti-viral drug, and a cholesterol lowering substance.

18. The composition as in claim 17, wherein the anti-inflammatory substance selected from at least one of ibuprofen, diclofenac, aspirin, and naproxen, the anti-degradation substance is selected from at least one of a glucosamine compound, a chondroitin compound, methylsulfonylmethane, and an omega-3 fatty acid, the anti-viral drug is selected from at least one of oseltamivir and zanamivir, and the cholesterol lowering substance is a statin.

19. The composition as in claim 1, further comprising a concentration of sesamin that provides a sesamin dosage of at least approximately 5 mg/day when the composition is consumed by the living organism.

20-40. (canceled)

41. A process for manufacturing a composition that therapeutically affects a pro-inflammatory cytokine condition within a living organism when consumed thereby, the process comprising:
providing the composition with xanthones of a concentration of between approximately 0.005 ug/ml and approximately 0.5 ug/ml.

42. (canceled)

43. The process as in claim 41, wherein the composition at least one of facilitates and effectuates decrease in quantity of the pro-inflammatory cytokine within the living organism by approximately 10% and approximately 50% when consumed by the living organism.

44. The process as in claim 41, further comprising:
providing the composition with sesamin of a concentration of at least approximately 0.001 mg/ml.

45. (canceled)

46. The process as in claim 41, wherein the composition further at least one of facilitates and effectuates an increase in quantity of an anti-inflammatory cytokine within the living organism.

47. The process as in claim 41, wherein the composition further at least one of facilitates and effectuates inhibition of a viral neuraminidase within the living organism to thereby provide a viral activity inhibitory effect.

48. The process as in claim 41, wherein the viral neuraminidase is a neuraminidase of an influenza virus, the influenza virus being one of influenza A (H1N1), influenza A (H2N2), influenza A (H3N2), and influenza A (H5N1) virus, inhibition of the influenza virus providing an anti-influenza effect within the living organism.

49. The process as in claim 41, further comprising:
providing a predetermined concentration of at least one of an anti-inflammatory substance, a connective tissue anti-degradation substance, and an anti-viral drug, and a cholesterol lowering substance.

50. The process as in claim 49, wherein the anti-inflammatory substance is selected from at least one of ibuprofen, diclofenac, aspirin, and naproxen, the anti-degradation substance is selected from at least one of a glucosamine compound, a chondroitin compound, methylsulfonylmethane, and an omega-3 fatty acid, the anti-viral drug is selected from at least one of oseltamivir and zanamivir, and the cholesterol lowering substance is a statin.

51. A composition comprising a predetermined concentration of xanthones to provide a xanthones dosage of at least approximately 5 mg/day when consumed by a living organism, the composition at least one of facilitating and effectuating decrease in quantity of a pro-inflammatory cytokine within the living organism when consumed thereby.

52. The composition as in claim 51, wherein the composition further comprises a predetermined concentration of sesamin to provide a sesamin dosage of at least approximately 5 mg/day when consumed by the living organism.

53. The composition as in claim 51, wherein the decrease in quantity of the pro-inflammatory cytokine within the living organism is at least approximately 10%.

54. The composition as in claim 51, wherein the composition further at least one of facilitates and effectuates increase in quantity of an anti-inflammatory cytokine within the living organism when consumed thereby.

55. The composition as in claim 51, wherein the composition further at least one of facilitates and effectuates inhibition of a viral neuraminidase within the living organism to thereby provide a viral activity inhibitory effect.

56. The composition as in claim 55, wherein the viral neuraminidase is a neuraminidase of an influenza virus, the influenza virus being one of influenza A (H1N1), influenza A (H2N2), influenza A (H3N2), and influenza A (H5N1) virus, inhibition of the influenza virus providing an anti-influenza effect within the living organism.

57. The composition as in claim 51, further comprising a predetermined concentration of at least one of an anti-inflammatory substance, a connective tissue anti-degradation substance, and an anti-viral drug, and a cholesterol lowering substance.

58. The composition as in claim 57, wherein the anti-inflammatory substance is selected from at least one of ibuprofen, diclofenac, aspirin, and naproxen, the anti-degradation substance is selected from at least one of a glucosamine compound, a chondroitin compound, methylsulfonylmethane, and an omega-3 fatty acid, the anti-viral drug is selected from at least one of oseltamivir and zanamivir, and the cholesterol lowering substance is a statin.
